(19) United States
(12) Patent Application Publication
(10) Pub. No.: US 2023/0391833 A1
(43) Pub. Date: Dec. 7, 2023

(54) MODIFIED VACCINE DESIGN DEVELOPMENTS

(71) Applicant: VLP Therapeutics Japan, INC., Tokyo (JP)

(72) Inventor: Wataru AKAHATA, Kensington, MD (US)

(73) Assignee: VLP Therapeutics Japan, INC., Tokyo (JP)

(21) Appl. No.: 18/325,281

(22) Filed: May 30, 2023

Related U.S. Application Data
(60) Provisional application No. 63/347,156, filed on May 31, 2022.

(51) Int. Cl. C07K 14/005; C07K 14/195; C07K 14/46

(52) U.S. Cl. CPC C07K 14/005; C07K 14/195; C07K 14/463; C07K 2319/02; C07K 2319/03; C12N 2760/16134; C12N 2770/36134; A61K 39/00

(57) ABSTRACT

Provided herein is an isolated polynucleotide, which encodes a polypeptide comprising an antigen protein fused to a signal sequence and a transmembrane domain, and optionally to ferritin. Also provided herein is an isolated polynucleotide, which encodes alphavirus non-structural proteins nsp1, nsp2, nsp3 and nsp4 and a polypeptide comprising an antigen protein fused to a signal sequence and a transmembrane domain, and optionally to ferritin. The antigen may be influenza. The polynucleotide such as RNA is useful as a vaccine against influenza infection.

### CROSS REFERENCE TO RELATED APPLICATIONS

This application claims the benefit of U.S. Provisional Patent Application No. 63/347,156 filed on May 31, 2022. The entire disclosures of those prior applications are hereby incorporated by reference.

### REFERENCE TO AN ELECTRONIC SEQUENCE LISTING

The contents of the electronic sequence listing (Q287594_sequence listing as filed.xml; Size: 36,225 bytes; and Date of Creation: May 26, 2023) is herein incorporated by reference in its entirety.

### BACKGROUND

### Technical Field

The present disclosure relates generally to the field of a novel vaccine design and to a method and a composition for treating and/or immunizing against antigen. In particular, the present disclosure relates to an influenza vaccine.

### Background Art

Influenza (flu) is a contagious respiratory illness caused by influenza viruses that infect the nose, throat, and lungs. Some people, such as older people, young children, and people with certain health conditions, are at higher risk of serious flu complications. There are two main types of influenza (flu) viruses: Types A and B. The influenza A and B viruses that routinely spread in people (human influenza viruses) are responsible for seasonal flu epidemics each year.

The first and most important step in preventing flu is to get a flu vaccine each year. Flu vaccine has been shown to reduce flu related illnesses and the risk of serious flu complications that can result in hospitalization or even death.

The different type of flu vaccines are available including quadrivalent Flu Vaccine that protect against four different flu viruses; High-Dose Flu Vaccine that the high dose vaccine contains 4 times the amount of antigen (the part of the vaccine that helps your body build up protection against flu viruses) as a regular flu shot and is licensed specifically for people 65 years and older; Cell-Based Flu Vaccines that Cell-based vaccines are grown in cultured cells of mammalian origin instead of in hens' eggs; Nasal Spray Flu Vaccine that Live Attenuated Influenza Vaccine [LAIV] is given as a nasal spray; Flu Vaccination via Jet Injector that is approved for use in people 18 through 64 years old; Adjuvanted Vaccine that adjuvanted flu vaccine is made with an ingredient added to a vaccine that helps create a stronger immune response and is licensed specifically for people 65 years and older; Recombinant Flu Vaccines that Recombinant flu vaccines are produced using a method that does not require an egg-grown vaccine virus (https://www.cdc.gov/flu/about/index.html). Data on vaccine effectiveness (VE) was obtained from the March CDC Morbidity and Mortality Weekly Report. VE was calculated using data from the 3,363 children and adults with an acute respiratory infection (ARI) enrolled in the US Influenza Vaccine Effectiveness Network across seven different study sites in the US from October 2021 to February 2022. AVE of only 16% was observed from the 2021-22 seasonal flu vaccine in protecting the US population from contracting the most common influenza virus in current circulation, A(H3N2). More specifically, VE against mild to moderate ARI associated with the influenza A(H3N2) virus in outpatients, who received medical attention, was 16%, which was not significantly effective. Further, VE against outpatients with ARI associated with influenza A virus types, who were medically attended, was even less significantly effective at 14%. The low vaccine efficacy in the most common influenza strain in circulation is particularly concerning (https://www.clinicaltrialsarena.com/comment/us-flu-vaccine-efficacy/).

More promising influenza vaccine with high efficacy for the long periods is expected.

### SUMMARY OF INVENTION

The present disclosure relates to novel antigenically-active proteins/polypeptides capable of inducing protection against an antigen. The protein/polypeptide disclosed herein include an antigen protein fused to a signal sequence and a transmembrane domain, and optionally to ferritin.

In another aspect, the present disclosure relates to a novel polynucleotide encoding the above discussed novel antigenically-active protein/polypeptide capable of inducing protection against the antigen.

In another aspect, the present disclosure relates to a novel alphavirus replicon (self-amplifying RNA “saRNA”) that can express the above discussed antigenically-active protein/polypeptide. The alphavirus replicon includes polynucleotide such as RNA encoding alphavirus non-structural proteins nsp1, nsp2, nsp3 and nsp4 and a polynucleotide encoding the above-discussed antigenically active protein/polypeptide as a gene of interest.

In yet another aspect, the present disclosure relates to a vaccine comprising the above discussed polypeptide or polynucleotide. Especially, the present disclosure provides a vaccine comprising a polynucleotide encoding a polypeptide comprising an antigen protein fused to a signal sequence and a transmembrane domain, and optionally to ferritin. The vaccine preferably comprises a saRNA comprising a polynucleotide encoding alphavirus non-structural proteins nsp1, nsp2, nsp3 and nsp4, and a polypeptide comprising antigen protein fused to a signal sequence and a transmembrane domain, and optionally to ferritin. In one preferred embodiment, the antigen is an influenza antigen. The vaccine can be used for preventing and/or treating a subject from influenza infection.

In yet another aspect, the present disclosure relates to a method for inducing and/or enhancing immune response against an antigen. In one preferred embodiment, the method for immunizing, preventing or treating a subject from influenza comprising administering an effective amount of the above-discussed polypeptide or polynucleotide, such as the saRNA to the subject in need thereof.

In still another aspect, the present disclosure relates to use of the above-discussed polypeptide or polynucleotide for the manufacture of a medicament.

In further aspect, the present disclosure relates to a novel polynucleotide encoding a polypeptide comprising an antigen protein fused to a signal sequence and a transmembrane domain, and optionally to ferritin. The polynucleotide may be a saRNA comprising a polynucleotide encoding alphavirus non-structural proteins nsp1, nsp2, nsp3 and nsp4, and a polypeptide comprising an antigen protein fused to a signal sequence and a transmembrane domain, and optionally to ferritin.

### BRIEF DESCRIPTION OF THE DRAWINGS

FIG. 1 A representative construct of a saRNA disclosed in this application.

FIG. 2 Schematic view of the constructs F1, F2, F4 and F5.

FIG. 3 Western blotting results of the lysate of the cells transfected with the saRNA.

FIG. 4 FACS analysis of the HA antigen expressed on the cells transfected with the saRNA.

### DETAILED DESCRIPTION

As used herein “influenza” is meant to refer to the family Orthomyxoviridae (a group of RNA viruses). Influenza viruses are categorized as types A, B, C, and D. These major types generally produce similar symptoms but are completely unrelated antigenically, so that infection with one type confers no immunity against the others. The A viruses cause the great influenza epidemics, and the B viruses cause smaller localized outbreaks. The C viruses cause only mild respiratory illness in humans. Influenza D viruses are not known to infect humans and have been observed only in pigs and cattle.

Influenza A viruses are classified into subtypes, and both influenza B and subtypes of influenza A are further divided into strains. Subtypes of influenza A are differentiated mainly on the basis of two surface antigens (foreign proteins)-hemagglutinin (H) and neuraminidase (N). Examples of influenza A subtypes include H1N1, H5N1, and H3N2. Influenza B viruses are subdivided into two major lineages, B/Yamagata and B/Victoria. Strains of influenza B and strains of influenza A subtypes are further distinguished by variations in genetic sequence.

“Influenza structural protein” used herein may be a naturally occurring virus structural protein or a modified protein thereof. The modified protein may be a fragment of the naturally occurring virus structural protein. In one embodiment, the modified protein has at least 70%, 75%, 80%, 85%, 90%, 95% or 98% amino acid sequence identity to a naturally occurring viral structural protein or its fragment. In one embodiment, the modified protein is a mutant where at most 10% of the amino acids are deleted, substituted, and/or added based on a naturally occurring viral envelope protein or its fragment.

As used herein, “transmembrane domain (TM)” is a protein derived either from a natural or from a synthetic source. Where the source is natural, the domain in some aspects is derived from any membrane-bound or transmembrane protein. In one aspect, the membrane-bound or transmembrane protein is a protein heterologous to Influenza. Examples of the membrane-bound or transmembrane proteins may include the alpha, beta or zeta chain of a T-cell receptor, CD28, CD3 epsilon, CD45, CD4, CD5, CDS, CD9, CD16, CD22, CD33, CD37, CD64, CD80, CD86, CD134, CD137, CD154; toll-like receptors (TLR) such as TLR1-TLR10 in human and TLR1-TLR9, TLR11-TLR13 in mouse; interleukin (IL) receptors such as IL-1-28 receptor, RANTES receptors (CCR1, CCR3, CCR5), MIP-1 receptor, PF4 receptor, M-CSF receptor and NAP-2 receptor belonging to GPCR chemokine receptor; hemagglutinin (HA). In another aspect, the membrane-bound or transmembrane protein is a protein derived from Influenza virus.

Examples of transmembrane proteins may also include the followings:

5-Lipoxygenase-Activating Protein, ABC Transporters, ACBP, Amyloid beta (A4), Bcl-2 Inhibitors, BNIPs, CAAX protease, Cytochromes P450, E-NPPs, EPHA1, EPHA2, EPHA3, EPHA4, Fatty Acid Desaturases, Gamma secretase, Glucose transporter, Glycophorins, GPCR, HER2/ErbB2, HER3/ErbB3, HER4/ErbB4, HSD-11β, Hypoxia-induced Proteins, Immunoglobulins, Insulin receptor, Integrins, Ion channel, MAPEG, MFS, MinK Family, MPPs, Peptidase AD, Peptidase Family M48, Peptidase MA, Protein Jagged, Receptor-type Kinases, SNARE Complex, Sulfatases, TNF receptor, Transmembrane Proteins 14, Transporter, TROBP, VEGF receptors, Aldehyde Dehydrogenases, Ammonia and Urea transporters, FMN-linked Oxidoreductases, Leucine Rich Repeat (LRR)-Containing Transmembrane Proteins, Leukotriene C4 synthase, Lysosome-associated membrane glycoprotein, Major Intrinsic Protein (MIP)/FNT superfamily, Microsomal prostaglandin E synthase, N-(deoxy)ribosyltransferase-like Membrane Proteins, Neutral/alkaline Ceramidases, Oligosaccharyl Transferase, Pentameric Ligand-gated Ion Channels, Rhodopsin-like receptors and pumps, Single-helix ATPase Regulators, Squalene/phytoene Synthase, Stearoyl -CoA desaturase 1, Stannin (SNN) Membrane Proteins, T-cell Surface Glycoprotein CD3 Zeta Chain, Tetratricopeptide repeat (TPR) Alpha-Helical Repeat Proteins, Transmembrane Proteins with NAD(P)-binding Rossmann-fold Domains.

In addition, monotypic/peripheral proteins that attached to the lipid bilayer or other integral proteins and peptide may also be used as transmembrane proteins. Examples may include Alpha/Beta-Hydrolase, Annexins, Bet V1-Like Protein, C1 Domain-Containing Protein, C2 Domain-containing Protein, CoA-Dependent Acyltransferases, CRAL-TRIO Domain-Containing Protein, DNase I-like protein, Fibrinogen, FYVE/PHD Zinc Finger Protein, Galactose-Binding Domain-Like Protein, Glycolipid Transfer Protein, Immunoglobulin-Like Superfamily (E Set) Protein, Lipocalin, Lipoxygenase, PGBD superfamily, PH Domain-Like Protein, Phosphatidylinositol 3-/4-Kinase, PLC-like Phosphodiesterase, Phosphotyrosine Protein Phosphatases II, P-Loop Containing Nucleoside Triphosphate Hydrolase, Protein kinase superfamily , PX Domain-Containing Protein, Saposin, Synuclein and Transcriptional factor tubby.

As used herein “ferritin” refers to any one or a combination of at least two of derived ferritin, amphibian-derived ferritin, bacterial-derived ferritin or plant-derived mammalian-ferritin. Mammalian-derived ferritin or bacterial-derived ferritin is preferred. Preferable mammalian-derived ferritin comprises any one or a combination of at least two of human-derived ferritin, murine-derived ferritin or horse spleen ferritin. Preferable amphibian-derived ferritin includes bullfrog. Preferable bacterial-derived ferritin includes Helicobacter pylori ferritin or Escherichia coli ferritin. Preferable source of the ferritin includes any one or a combination of at least two of natural extraction products, artificial synthesis products or genetic engineering technology products. In one embodiment, Helicobacter pylori (H. pylori)-bullfrog hybrid ferritin, for example, Helicobacter pylori-bullfrog hybrid ferritin constructed by fusing residues 2-9 of bullfrog (Rana catesbeiana) ferritin lower subunit (UniProt: P07797 with a N8Q mutation to abolish a potential N-glycosylation site) to H. pylori nonheme ferritin (UniProt: Q9ZLI1, residues 3-167) with an I7E mutation to preserve the conserved salt bridge found in human and bullfrog ferritins (6R and 14E in both human light chain and bullfrog lower-subunit ferritins) with 6R of bullfrog ferritin (Kanekiyo et al., Cell. 2015 Aug. 27; 162(5): 1090-1100) may be employed.

As used herein, “nucleoside” refers to a molecule consisting of a guanine (G), adenine (A), thymine (T), uridine (U), cytidine (C) or a modified nucleoside thereof.

A modified nucleoside includes, but not limited to, pseudouridine, N1-methyl-pseudouridine, 5-methyl-uridine, pseudoucytidine, N1-methyl-pseudocytidine and 5-methyl-cytidine.

Pseudouridine or pseudocytidine is an isomer of the uridine or cytidine in which the uracil or cytosine is attached via a carbon-carbon instead of a nitrogen-carbon glycosidic bond.

In one embodiment, the modified nucleoside is independently selected from N1-methyl-pseudouridine or 5-methyl-cytidine. In one embodiment, the mRNA or saRNA includes substantially 100% modified cytidine (e.g. 5-methyl-cytidine) and 100% modified uridine (e.g. N1-methyl-pseudouridine), 100% modified cytidine and 80% modified uridine, and 50% modified cytidine and 50% modified uridine. In one embodiment, saRNA includes less than 100% modified uridine.

The expression “transmembrane domain” used in the present disclosure includes at least transmembrane region(s) of the membrane-bound or transmembrane protein. In addition, the transmembrane domain may also include juxtamembrane domain (JMD) and/or cytoplasmic tail of the membrane-bound or transmembrane protein.

Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

Preferable transmembrane domain may be those derived from influenza virus hemagglutinin (HA), CD80, Toll-like receptor 4(TLR4). Specific examples may include a protein consisting of the transmembrane domain and the cytoplasmic tail of Influenza virus hemagglutinin “HA (TM/CT)”; a protein consisting of transmembrane domain and cytoplasmic tail of human CD80; a protein consisting of transmembrane domain(TM) and Toll/interleukin-1 receptor domain (TIR), and a protein consisting of the juxtamembrane domain (JMD).

As used herein, “signal sequence” (sometimes referred to as signal peptide, targeting signal, localization signal, localization sequence, transit peptide, leader sequence or leader peptide) is a polynucleotide or polypeptide, depending on the context. Signal sequence is from about 9 to 200 nucleotides or 3-70 amino acids in length that, optionally, is incorporated at the 5′ or N-terminus of the coding region or the protein. Some signal sequences are cleaved from the protein, for example by a signal peptidase after the proteins are transported to the desired site.

The signal sequence is not limited and may be selected from various sequences. In some embodiment, the signal sequence may be of the target protein to be expressed by the alphavirus replicon.

In some embodiments, the signal sequence of influenza HA, COVID-19 or IL-2, and especially influenza HA may be employed.

The influenza structural protein, the transmembrane domain and/or the signal sequence may be directly or indirectly fused. In one embodiment, one or two linkers may intervene between them.

Also the influenza structural protein, the transmembrane domain and/or the signal sequence can be truncated and replaced by short linkers. In some embodiments, the viral structural protein, the transmembrane domain and/or the signal sequence include one or more peptide linkers.

The antigen protein, the signal sequence, the transmembrane domain and optionally, ferritin may further be fused to a T cell epitope. “T cell epitope” may be a CD4+ T-cell target epitope, CD8+ T cell target epitope or a Pan-DR epitope (PADRE). As used herein, T cell epitope may be derived from the virus to be treated.

The expression “PADRE” used in the present disclosure means Pan HLA DR-binding epitope, a peptide that universally activates antigen specific-CD4+ T cells. The amino acid sequence of PADRE is AKFVAAWTLKAAA.

The viral structural protein, a signal sequence and transmembrane domain, and optionally ferritin and at least one universal epitope such as PADRE may be directly or indirectly fused. In one embodiment, one or two linkers may intervene between them.

Also the viral structural protein, a signal sequence and transmembrane domain and optionally ferritin and at least one or more T-cell target epitopes can be truncated and replaced by short linkers. In some embodiments, the viral structural protein, the transmembrane domain and/or the signal sequence include one or more peptide linkers.

An example of a short linker consists of from 2 to 25 amino acids (e.g. 2, 3, 4, 5 or 6 amino acids). Usually, it is from 2 to 15 amino acids in length, such as SG, GS, SGG, GGS SGSG and TRGGS. In certain circumstances, the linker can consist of only one amino acid, such as glycine(G), serine (S) and cysteine (C).

When the influenza structural protein is chemically conjugated to the transmembrane domain and/or the signal sequence through a chemical cross-linker, examples of the cross-linkers include, but are not limited to, SMPH, Sulfo-MBS, Sulfo-EMCS, Sulfo-GMBS, Sulfo-SIAB, Sulfo-SMPB, Sulfo-SMCC, SVSB, and SIA. Chemical cross-linkers available on the market may also be employed.

IgG-derived substances can also be used as a linker. Examples of IgG-derived substances include IgG1 to IgG4 comprising (i) full (hinge-CH₂CH₃) (ii) half (hinge-CH₃) and (iii) short (12aa hinge only). Preferable example is IgG4-CH₃.

By “alphavirus structural protein” is meant a polypeptide or fragment thereof having at least about 80% amino acid sequence identity to a naturally occurring viral capsid or envelope protein. In one embodiment, the alphavirus structural protein has at least about 85%, 90%, 95% or greater amino acid sequence identity with Eastern Equine Encephalitis Virus (EEEV), Venezuelan Equine Encephalitis Virus (VEEV), Everglades Virus, Mucambo Virus, Pixuna Virus, Western Equine Encephalitis Virus (WEEV), Sindbis Virus, Semliki Forest Virus, Middleburg Virus, Chikungunya Virus (CHIKV), O′nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, or Buggy Creek Virus. Wild type amino acid sequences of alphavirus structural proteins can be obtained from GenBank.

By “alphavirus structural protein” is meant a polypeptide or fragment thereof having at least about 80% amino acid sequence identity to a naturally occurring viral capsid or envelope protein. In one embodiment, the alphavirus structural protein has at least about 85%, 90%, 95% or greater amino acid sequence identity with Eastern Equine Encephalitis Virus (EEEV), Venezuelan Equine Encephalitis Virus (VEEV), Everglades Virus, Mucambo Virus, Pixuna Virus, Western Equine Encephalitis Virus (WEEV), Sindbis Virus, Semliki Forest Virus, Middleburg Virus, Chikungunya Virus (CHIKV), O′nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J virus, Fort Morgan Virus, Ndumu Virus, or Buggy Creek Virus. Wild type amino acid sequences of alphavirus structural proteins can be obtained from GenBank.

In specific embodiments, the alphavirus is a CHIKV, for example CHIKV strain 37997 or LR2006 OPY-1. In other embodiments, the alphavirus is a VEEV, for example VEEV strain TC-83.

By “alphavirus replicon” is meant an RNA molecule which can direct its own amplification in vivo in a target cell. The replicon encodes the polymerase(s) which catalyze RNA amplification (nsp1, nsp2, nsp3, nsp4) and contains cis RNA sequences required for replication which are recognized and utilized by the encoded polymerase(s). An alphavirus replicon typically contains the following ordered elements: 5′ UTR, sequences which encode alphavirus nonstructural proteins (nsp1, nsp2, nsp3, nsp4), 3′ UTR, and a poly A signal. An alphavirus replicon also contains one or more viral sub-genomic promoters directing the expression of the gene of interest. Those sequences may have one or more mutations taught in prior art references.

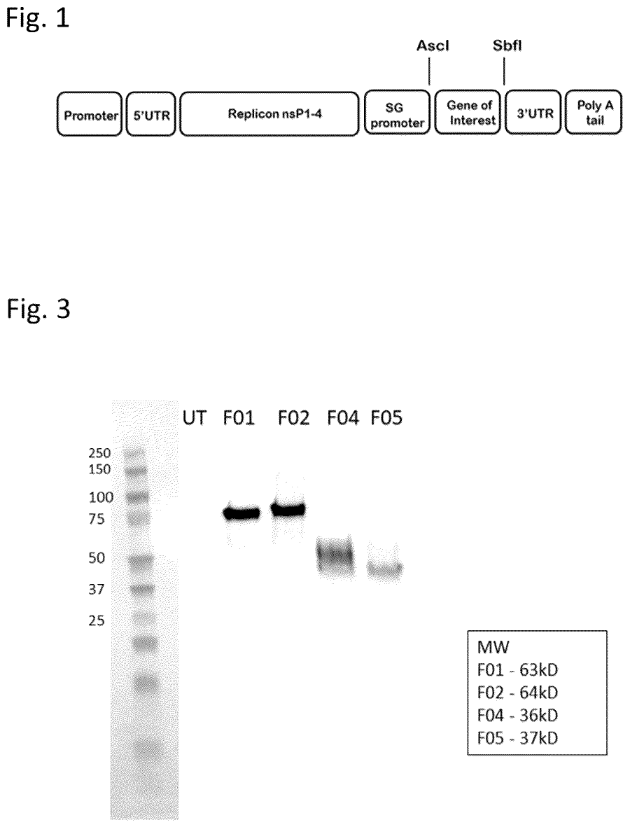

The alphavirus replicon provided by the present disclosure may have the construct shown in FIG. 1.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e<″3> and e<″100> indicating a closely related sequence.

By “effective amount” is meant the amount of an agent required to ameliorate the symptoms of a disease relative to an untreated patient. The effective amount of active compound(s) used to practice the present invention for prevention or treatment of a disease varies depending upon the manner of administration, the age, body weight, and general health of the subject. Ultimately, the attending physician or veterinarian will decide the appropriate amount and dosage regimen. Such amount is referred to as an “effective” amount.

A satisfactory effect can be obtained by systemic administration, e.g. intramuscular administration, subcutaneous administration or intravenous administration 1-4 times at the amount of 10³-10¹⁰ Infectious Unit (IU) or 0.01-500 μg per time, preferably 10⁵-10¹⁰ IU or 0.1-100 μg per time, for example 10⁷-10⁹ IU or 1-50 μg per one time. The replicon may preferably be formulated in a vaccine composition suitable for administration in a conventional manner.

By “subject” is meant a mammal, including, but not limited to, a human or non-human mammal, such as a bovine, equine, canine, ovine, or feline.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive.

Unless specifically stated or obvious from context, as used herein, the terms “a”, “an”, and “the” are understood to be singular or plural.

In this specification and claims, the term “about” covers a ±20%, ±10% or ±5% value of the associated numerical value.

The art will acknowledge that polynucleotide sequences described in the specification and claims will recite “T”s in a representative DNA sequence but where the sequence represents RNA, the “T”s would be substituted for “U”s.

Any vaccine compositions or methods provided herein can be combined with one or more of any of the other vaccine compositions and methods provided herein.

The term “vector” refers to the means by which a nucleic acid sequence can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids or bacmids.

Typically, the nucleic acid molecule to be expressed is “operably linked” to a promoter and/or enhancer, and is subject to transcription regulatory control by the promoter and/or enhancer.

The method of transfection and the choice of expression vehicle will depend on the host system selected. Transfection methods are described, e.g., in Ausubel et al. (supra); expression vehicles may be chosen from those provided, e.g., in Cloning Vectors: A Laboratory Manual (P. H. Pouwels et al., 1985, Supp. 1987). The references cited in this paragraph are herein incorporated by reference.

A variety of expression systems exist for the production of the constructs of the invention. Expression vectors useful for producing the constructs include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as alphavirus (e.g. Chikungunya Virus (CHIKV) and Venezuelan Equine Encephalitis Virus (VEEV)), baculoviruses, papova viruses, such as SV40, vaccinia viruses, adenoviruses, fowl pox viruses, pseudorabies viruses and retroviruses, and vectors derived from combinations thereof.

Constructs and/or vectors used herein comprise alphavirus polynucleotides encoding nonstructural proteins nsp1, nsp2, nsp3 and nsp4 and a gene of interest encoding a polypeptide comprising an antigen such as a viral structural protein fused to a signal sequence and a transmembrane domain as discussed above. Specific example of the construct or vector is that shown in FIG. 1.

The vector may be, for example, a phage, plasmid, viral, or retroviral vector. The constructs and/or vectors that comprise the nucleotides should be operatively linked to an appropriate promoter, such as the CMV promoter, phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBacl pWINEO, pSV2CAT, pOG44, pXT1 and pSG, pSVK3, pBPV, pMSG, and pSVL. Other suitable vectors will be readily apparent to the skilled artisan.

Recombinant constructs can be prepared and used to transfect, can express viral proteins, including those described herein, into eukaryotic cells and/or prokaryotic cells. Thus, in one embodiment, the present disclosure provides host cells which comprise a vector (or vectors) that contain nucleic acids which encode alphavirus structural proteins, including capsid, E3, E2, 6K, and E1 or portions thereof, and a vector that comprises nucleic acids which encode alphavirus nsp1, nsp2, nsp3 and nsp4, and at least one of viral gene of interest under conditions which allow the formation of alphavirus replicon particle.

In one embodiment, said vector is a recombinant baculovirus. In another embodiment, said recombinant baculovirus is transfected into an insect cell. In a preferred embodiment, said cell is an insect cell. In another embodiment, said insect cell is a Sf9 cell.

One particular bacterial expression system for polypeptide production is the E. coli pET expression system (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved by using host strains that express T7 RNA polymerase in response to IPTG induction. Once produced, a recombinant polypeptide is then isolated according to standard methods known in the art, for example, those described herein.

Depending on the vectors and host cells selected, the constructs are produced by growing host cells transfected by the vectors under conditions whereby the recombinant proteins are expressed and the alphavirus replicon is generated, and constructs containing alphavirus replicon being packaged with the particle of alphavirus structural proteins are formed. In one embodiment, the invention comprises a method of producing a construct, that involves co-transfecting a vector comprising a polynucleotide encoding alphavirus non-structural protein nsp1, nsp2, nsp3 and nsp4, and at least one gene of interest encoding the polypeptide comprising a viral structural protein fused to a signal sequence and/or transmembrane domain, and at least one vector each encoding at least one alphavirus structural protein into suitable host cells and expressing said alphavirus structural protein under conditions that allow construct formation. In another embodiment, the eukaryotic cell is selected from the group consisting of, yeast, insect, amphibian, avian or mammalian cells. The selection of the appropriate growth conditions is within the skill or a person with skill of one of ordinary skill in the art.

Methods to grow cells that produce alphavirus replicon particles of the invention include, but are not limited to, batch, batch-fed, continuous and perfusion cell culture techniques. In one embodiment, cells co-transfected with a vector encoding an alphavirus replicon and a vector comprising a polypeptide encoding capsid, and a vector comprising a polynucleotide encoding envelope proteins, such as those derived from a CHIKV or VEEV are grown in a bioreactor or fermentation chamber where cells propagate and express protein (e.g., recombinant proteins) for purification and isolation. Typically, cell culture is performed under sterile, controlled temperature and atmospheric conditions. A bioreactor is a chamber used to culture cells in which environmental conditions such as temperature, atmosphere, agitation and/or pH can be monitored. In one embodiment, the bioreactor is a stainless steel chamber. In another embodiment, said bioreactor is a pre-sterilized plastic bag (e.g., Cellbag.®TM., Wave Biotech, Bridgewater, N.J., the contents of the cited document is herein incorporated by reference). In other embodiment, said pre-sterilized plastic bags are about 10 L to 1000 L bags.

In another embodiment, an RNA molecule such as an alphavirus replicon may be generated by conventional procedures known to the art from a template DNA sequence. In vitro transcription (IVT) methods permit template-directed synthesis of RNA molecules. IVT methods permit synthesis of large quantities of RNA transcript. Generally, IVT utilizes a DNA template comprising a promoter sequence upstream of a sequence of interest. The promoter sequence is most commonly of bacteriophage origin such as the T7, T3 or SP6 promoter sequence but many other promotor sequences can be tolerated including those designed de novo. Transcription of the DNA template is typically best achieved by using the RNA polymerase corresponding to the specific bacteriophage promoter sequence. Exemplary RNA polymerases include, but are not limited to T7 RNA polymerase, T3 RNA polymerase, or SP6 RNA polymerase, among others. IVT is generally initiated at a dsDNA but can proceed on a single strand. Kits for in vitro transcription such as T7 transcription kit (RiboMax™ Express Large Scale RNA production System, Promega (WI USA)).

As used herein, the term “pharmaceutically acceptable carrier” means one or more compatible solid or liquid fillers, diluents or encapsulating substances which are suitable for administration to a human or other vertebrate animal, including any and all aqueous solvents (e.g., water, alcoholic/aqueous solutions, saline solutions, parenteral vehicles, such as sodium chloride, and Ringer's dextrose), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a vaccine composition are adjusted according to well-known parameters.

Encapsulating substances refers to a delivery vehicle where the polynucleotide or vector is packaged, such as a replicon particle (e.g. the alphavirus replicon particle described in US patent publication No. 2019-0185822, the contents of the document is incorporated by reference) and a lipid delivery system (e.g. liposome).

In some embodiments, the vaccine compositions or formulations of the present disclosure comprise a lipid delivery system, e.g., a liposome, a lioplexes, a lipid nanoparticle, or any combination thereof. The polynucleotides such as an alpha virus replicon described herein can be formulated using one or more liposomes, lipoplexes, or lipid nanoparticles. Liposomes, lipoplexes, or lipid nanoparticles can be used to improve the efficacy of the polynucleotides directed protein production as these formulations can increase cell transfection by the polynucleotide; and/or increase the translation of encoded protein. The liposomes, lipoplexes, or lipid nanoparticles can also be used to increase the stability of the polynucleotides.

Liposomes are artificially-prepared vesicles that may primarily be composed of a lipid bilayer and may be used as a delivery vehicle for the administration of pharmaceutical formulations. Liposomes can be of different sizes. A multilamellar vesicle (MLV) may be hundreds of nanometers in diameter, and may contain a series of concentric bilayers separated by narrow aqueous compartments. A small unicellular vesicle (SUV) may be smaller than 50 nm in diameter, and a large unilamellar vesicle (LUV) may be between 50 and 500 nm in diameter. Liposome design may include, but is not limited to, opsonins or ligands to improve the attachment of liposomes to unhealthy tissue or to activate events such as, but not limited to, endocytosis. Liposomes may contain a low or a high pH value in order to improve the delivery of the pharmaceutical formulations.

The formation of liposomes may depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

In some embodiments, the polynucleotides such as alpha virus replicon described herein may be encapsulated by the liposome and/or it may be contained in an aqueous core that may then be encapsulated by the liposome.

In some embodiments, the polynucleotides such as alpha virus replicon described herein can be formulated in a cationic oil-in-water emulsion where the emulsion particle comprises an oil core and a cationic lipid that can interact with the polynucleotide anchoring the molecule to the emulsion particle. In some embodiments, the polynucleotides described herein can be formulated in a water-in-oil emulsion comprising a continuous hydrophobic phase in which the hydrophilic phase is dispersed.

In some embodiments, the polynucleotides such as alpha virus replicon described herein can be formulated in a lipid-polycation complex. As a non-limiting example, the polycation can include a cationic peptide or a polypeptide such as, but not limited to, polylysine, polyornithine and/or polyarginine and the cationic peptides.

In some embodiments, the polynucleotides such as alpha virus replicon described herein can be formulated in a lipid nanoparticle (LNP).

Lipid nanoparticle formulations typically comprise one or more lipids. In some embodiments, the lipid is a cationic or an ionizable lipid. In some embodiments, lipid nanoparticle formulations further comprise other components, including a phospholipid, a structural lipid, a quaternary amine compound, and a molecule capable of reducing particle aggregation, for example a PEG or PEG-modified lipid. In some embodiments, the amount of the cationic and ionizable lipids in the lipid composition ranges from about 0.01 mol % to about 99 mol %.

LNPs contain a pH-sensitive ionizable cationic lipid that attract anionic nucleic acids to form the core of self-assembling nanoparticle to ensure high encapsulation. At physiological pH, LNPs are neutral, eliminating a mechanism of toxicity seen with permanently cationic molecules.

These same pH-sensitive lipids are responsible for responding to the acidic environment of the endosome and triggering the disruption of the endosome and release of the nucleic acid into the cell.

This replicon based vaccine technology is a unique platform technology for the vaccination as a RNA can self-amplify to produce the vaccine antigen and deliver into the cellular organ. Moreover, this replicon based vaccine technology overcomes the challenges commonly associated with DNA based vaccines, such as risk of genome integration or the high doses and devices needed for administration, e.g. electroporation, and expects the higher immunogenicity with minimum dose based on the self-replication system over the mRNA technology.

According to the present invention, novel antigenically-active proteins/polypeptides are also useful for producing antibodies for diagnosis and protecting against antigens while minimizing the possibility of ADE. The proteins/polypeptides disclosed herein include minimum sequences encoding the RBD fused to a signal sequence and/or to a transmembrane domain (TMD) sequence, intended to maximize immunogenicity and minimize ADE.

The invention will be described in detail with reference to the following examples, which, however, are not intended to limit the scope of the present application.

### EXAMPLE 1

Each gene encoding shown below constructs 1-12 was synthesized by Integrated DNA Technologies, Inc. (https://www.idtdna.com/pages).

1. Construct 1 [symbol] [symbol]
sg [symbol]
(SEQ ID NO: 1)
MKAILVVLLYTFATANALHLGKCNIAGWILGNPECESLSTASSWSYIVET
PSSDNGTCYPGDFIDYEELREQLSSVSSFERFEIFPKTSSWPNHDSNKGV
TAACPHAGAKSFYKNLIWLVKKGNSYPKLSKSYINDKGKEVLVLWGIHHP
STSADQQSLYQNADAYVFVGSSRYSKKFKPEIAIRPKVRDQEGRMNYYWT
LVEPGDKITFEATGNLVVPRYAFAMERNAGsgESQVRQQFskdiekllne
qvnkemqssnlymsmsswcythsldgaglflfdhaaeeyehakkliifln
ennvpvqltsisapehkfegltqifqkayeheghisesinnivdhaiksk
dhatfnflqwyvaeqheeevlfkdildkielignenhglyladqyvkgia
ksrks*
2. Construct 2

[symbol]

[symbol]

[symbol]

(SEQ ID NO: 2)
MKAILVVLLYTFATANALHLGKCNIAGWILGNPECESLSTASSWSYIVET
PSSDNGTCYPGDFIDYEELREQLSSVSSFERFEIFPKTSSWPNHDSNKGV
TAACPHAGAKSFYKNLIWLVKKGNSYPKLSKSYINDKGKEVLVLWGIHHP
STSADQQSLYQNADAYVFVGSSRYSKKFKPEIAIRPKVRDQEGRMNYYWT
LVEPGDKITFEATGNLVVPRYAFAMERNAGGVKLESMGIYQILAIYSTVA
SSLVLLVSLGAISFWMCSNGSLQCRICI*
3. Construct 3

[symbol]

[symbol]

[symbol]

(SEQ ID NO: 3)
MKAILVVLLYTFATANALHLGKCNIAGWILGNPECESLSTASSWSYIVET
PSSDNGTCYPGDFIDYEELREQLSSVSSFERFEIFPKTSSWPNHDSNKGV
TAACPHAGAKSFYKNLIWLVKKGNSYPKLSKSYINDKGKEVLVLWGIHHP
STSADQQSLYQNADAYVFVGSSRYSKKFKPEIAIRPKVRDQEGRMNYYWT
LVEPGDKITFEATGNLVVPRYAFAMERNAGGVKLESTRIYQILAIYSTVA
SSLVLVVSLGAISFWMCSNGSLQCRICI*
4. Construct 4 [symbol] [symbol]
sg [symbol]
(SEQ ID NO: 4)
mfvflvllplvssLHLGKCNIAGWILGNPECESLSTASSWSYIVETPSSD
NGTCYPGDFIDYEELREQLSSVSSFERFEIFPKTSSWPNHDSNKGVTAAC
PHAGAKSFYKNLIWLVKKGNSYPKLSKSYINDKGKEVLVLWGIHHPSTSA
DQQSLYQNADAYVFVGSSRYSKKFKPEIAIRPKVRDQEGRMNYYWTLVEP
GDKITFEATGNLVVPRYAFAMERNAGsgESQVRQQFskdiekllneqvnk
emqssnlymsmsswcythsldgaglflfdhaaeeyehakkliiflnennv
pvqltsisapehkfegltqifqkayeheqhisesinnivdhaikskdhat
fnflqwyvaeqheeevlfkdildkielignenhglyladqyvkgiaksrk
s
5. Construct 5

[symbol]

[symbol]

[symbol]

(SEQ ID NO: 5)
mfvflvllplvssLHLGKCNIAGWILGNPECESLSTASSWSYIVETPSSD
NGTCYPGDFIDYEELREQLSSVSSFERFEIFPKTSSWPNHDSNKGVTAAC
PHAGAKSFYKNLIWLVKKGNSYPKLSKSYINDKGKEVLVLWGIHHPSTSA
DQQSLYQNADAYVFVGSSRYSKKFKPEIAIRPKVRDQEGRMNYYWTLVEP
GDKITFEATGNLVVPRYAFAMERNAGGVKLESMGIYQILAIYSTVASSLV
LLVSLGAISFWMCSNGSLQCRICI
6. Construct 6 [symbol] [symbol]
R

[symbol]

[symbol]

(SEQ ID NO: 6)
MKAILVVLLYTFATANADTLCIGYHANNSTDTVDTVLEKNVTVTHSVNLL
EDKHNGKLCKLRGVAPLHLGKCNIAGWILGNPECESLSTASSWSYIVETP
SSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPS
TSADQQSLYQNADAYVFVGSSRYSKKFKPEIAIRPKVRDQEGRMNYYWTL
VEPGDKITFEATGNLVVPRYAFAMERNAGSGIIISDTPVHDCNTTCQTPK
GAINTSLPFQNIHPITIGKCPKYVKSTKLRLATGLRNIPSIQSRGLFGAI
AGFIEGGWTGMVDGWYGYHHQNEQGSGYAADLKSTQNAIDEITNKVNSVI
EKMNTQFTAVGKEFNHLEKRIENLNKKVDDGFLDIWTYNAELLVLLENER
TLDYHDSNVKNLYEKVRSQLKNNAKEIGNGCFEFYHKCDNTCMESVKNGT
YDYPKYSEEAKLNREEIDGVKLESMGIYQILAIYSTVASSLVLLVSLGAI
SFWMCSNGSLQCRICI*
7. Construct 7 [symbol] [symbol]
R [symbol] sg [symbol]
(SEQ ID NO: 7)
MKAILVVLLYTFATANADTLCIGYHANNSTDTVDTVLEKNVTVTHSVNLL
EDKHNGKLCKLRGVAPLHLGKCNIAGWILGNPECESLSTASSWSYIVETP
SSDNGTCYPGDFIDYEELREQLSSVSSFERFEIFPKTSSWPNHDSNKGVT
AACPHAGAKSFYKNLIWLVKKGNSYPKLSKSYINDKGKEVLVLWGIHHPS
TSADQQSLYQNADAYVFVGSSRYSKKFKPEIAIRPKVRDQEGRMNYYWTL
VEPGDKITFEATGNLVVPRYAFAMERNAGSGIIISDTPVHDCNTTCQTPK
GAINTSLPFQNIHPITIGKCPKYVKSTKLRLATGLRNIPSIQSRGLFGAI
AGFIEGGWTGMVDGWYGYHHQNEQGSGYAADLKSTQNAIDEITNKVNSVI
EKMNTQFTAVGKEFNHLEKRIENLNKKVDDGFLDIWTYNAELLVLLENER
TLDYHDSNVKNLYEKVRSQLKNNAKEIGNGCFEFYHKCDNTCMESVKNGT
YDYPKYSEEAKLNREEIDsgESQVRQQFskdiekllneqvnkemqssnly
msmsswcythsldgaglflfdhaaeeyehakkliiflnennvpvqltsis
apehkfegltqifqkayeheqhisesinnivdhaikskdhatfnflqwyv
aeqheeevlfkdildkielignenhglyladqyvkgiaksrk
Constructs 8-14
Constructs 8-14 correspond to Constructs 1-7
respectively which are further fused to PADRE
(T epitope) at the C-terminal. (SEQ ID NO: 8-14)
Influenza A virus HA1:
(SEQ ID NO: 15)
DTLCIGYHANNSTDTVDTVLEKNVTVTHSVNLLEDKHNGKLCKLRGVAPL
HLGKCNIAGWILGNPECESLSTASSWSYIVETPSSDNGTCYPGDFIDYEE
LREQLSSVSSFERFEIFPKTSSWPNHDSNKGVTAACPHAGAKSFYKNLIW
LVKKGNSYPKLSKSYINDKGKEVLVLWGIHHPSTSADQQSLYQNADAYVF
VGSSRYSKKFKPEIAIRPKVRDQEGRMNYYWTLVEPGDKITFEATGNLVV
PRYAFAMERNAGSGIIISDTPVHDCNTTCQTPKGAINTSLPFQNIHPITI
GKCPKYVKSTKLRLATGLRNIPSIQS
Influenza A virus HA2:
(SEQ ID NO: 16)
GLFGAIAGFIEGGWTGMVDGWYGYHHQNEQGSGYAADLKSTQNAIDEITN
KVNSVIEKMNTQFTAVGKEFNHLEKRIENLNKKVDDGFLDIWTYNAELLV
LLENERTLDYHDSNVKNLYEKVRSQLKNNAKEIGNGCFEFYHKCDNTCME
SVKNGTYDYPKYSEEAKLNREEID
COVID-19 Signal Sequence:
(SEQ ID NO: 17)
MFVFLVLLPLVSS
Influenza A virus HA1 Signal Sequence:
(SEQ ID NO: 18)
MKAILVVLLYTFATANA
Influenza A virus HA1 head:
(SEQ ID NO: 19)
LHLGKCNIAGWILGNPECESLSTASSWSYIVETPSSDNGTCYPGDFIDYE
ELREQLSSVSSFERFEIFPKTSSWPNHDSNKGVTAACPHAGAKSFYKNLI
WLVKKGNSYPKLSKSYINDKGKEVLVLWGIHHPSTSADQQSLYQNADAYV
FVGSSRYSKKFKPEIAIRPKVRDQEGRMNYYWTLVEPGDKITFEATGNLV
VPRYAFAMERNAG
sg: Linker
TM/CT derived from Influenza A virus (A/Puerto
Rico/8/1934(H1N1)):
(SEQ ID NO: 20)
GVKLESMGIYQILAIYSTVASSLVLLVSLGAISFWMCSNGSLQCRICI
Or
TM/CT derived from influenza A virus
(A/California/07/2009 (H1N1)):
(SEQ ID NO: 21)
GVKLESTRIYQILAIYSTVASSLVLVVSLGAISFWMCSNGSLQCRICI
Ferritin (Helicobacter pylori-bullfrog
hybrid ferritin):
(SEQ ID NO: 22)
ESQVRQQFSkdiekllneqvnkemqssnlymsmsswcythsldgaglflf
dhaaeeyehakkliiflnennvpvqItsisapehkfegltqifqkayehe
qhisesinnivdhaikskdhatfnflqwyvaeqheeevlfkdildkieli
gnenhglyladqyvkgiaksrks
PADRE:
(SEQ ID NO: 23)
AKFVAAWTLKAAA

### EXAMPLE 2

### Preparation of Replicon Vector

Schematic construct of the alphavirus replicon is shown in FIG. 1. As the promoter in FIG. 1, T7 promoter was used.

VEEV full length replicon plasmid vector was prepared by the procedure disclosed in WO 2019/124441. Each of constructs 1-14 prepared in Example 1 was used as the gene of interest. Nucleotides encoding the construct was cloned into the VEEV replicon vector under the control of SG promoter. The VEEV replicon plasmid encoding each fragment was created by inserting AscI and SbfI restriction sites to obtain the full-length VEEV TC-83 replicon plasmid.

Nucleotide sequences of SG promoter, 5′UTR, 3′UTR and Poly A tail are as follows. RNA sequences were obtained by using those DNA sequences as template.

SG promoter:
(SEQ ID NO: 24)
cctgaatggactacgacatagtctagtccgccaag
5′UTR:
(SEQ ID NO: 25)
ataggcggcgcatgagagaagcccagaccaattacctacccaaa
3′UTR:
(SEQ ID NO: 26)
gcgatcgcatacagcagcaattggcaagctgcttacatagaactcgcggc
gattggcatgccgccttaaaatttttattttatttttcttttcttttccg
aatcggattttgtttttaatatttc
Poly A tail:
(SEQ ID NO: 27)
aaaaaaaaaaaaaaaaaaaaaaaaaaaaaaaaaaaaaaaaaaaaaaaaaa
aaaaa

VEEV TC-83 Replicon nsP1-4 amino acid sequence is as follows.

(SEQ ID NO: 28)
MEKVHVDIEEDSPFLRALQRSFPQFEVEAKQVTDNDHANARAFSHLASKL
IETEVDPSDTILDIGSAPARRMYSKHKYHCICPMRCAEDPDRLYKYATKL
KKNCKEITDKELDKKMKELAAVMSDPDLETETMCLHDDESCRYEGQVAVY
QDVYAVDGPTSLYHQANKGVRVAYWIGFDTTPFMFKNLAGAYPSYSTNWA
DETVLTARNIGLCSSDVMERSRRGMSILRKKYLKPSNNVLFSVGSTIYHE
KRDLLRSWHLPSVFHLRGKQNYTCRCETIVSCDGYVVKRIAISPGLYGKP
SGYAATMHREGFLCCKVTDTLNGERVSFPVCTYVPATLCDQMTGILATDV
SADDAQKLLVGLNQRIVVNGRTQRNTNTMKNYLLPVVAQAFARWAKEYKE
DQEDERPLGLRDRQLVMGCCWAFRRHKITSIYKRPDTQTIIKVNSDFHSF
VLPRIGSNTLEIGLRTRIRKMLEEHKEPSPLITAEDIQEAKCAADEAKEV
REAEELRAALPPLAADFEEPTLEADVDLMLQEAGAGSVETPRGLIKVTSY
AGEDKIGSYAVLSPQAVLKSEKLSCIHPLAEQVIVITHSGRKGRYAVEPY
HGKVVVPEGHAIPVQDFQALSESATIVYNEREFVNRYLHHIATHGGALNT
DEEYYKTVKPSEHDGEYLYDIDRKQCVKKELVTGLGLTGELVDPPFHEFA
YESLRTRPAAPYQVPTIGVYGVPGSGKSGIIKSAVTKKDLVVSAKKENCA
EIIRDVKKMKGLDVNARTVDSVLLNGCKHPVETLYIDEAFACHAGTLRAL
IAIIRPKKAVLCGDPKQCGFFNMMCLKVHFNHEICTQVFHKSISRRCTKS
VTSVVSTLFYDKRMRTTNPKETKIVIDTTGSTKPKQDDLILTCFRGWVKQ
LQIDYKGNEIMTAAASQGLTRKGVYAVRYKVNENPLYAPTSEHVNVLLTR
TEDRIVWKTLAGDPWIKILTAKYPGNFTATIEEWQAEHDAIMRHILERPD
PTDVFQNKANVCWAKALVPVLKTAGIDMTTEQWNTVDYFETDKAHSAEIV
LNQLCVRFFGLDLDSGLFSAPTVPLSIRNNHWDNSPSPNMYGLNKEVVRQ
LSRRYPQLPRAVATGRVYDMNTGTLRNYDPRINLVPVNRRLPHALVLHHN
EHPQSDFSSFVSKLKGRTVLVVGEKLSVPGKKVDWLSDQPEATFRARLDL
GIPGDVPKYDIVFINVRTPYKYHHYQQCEDHAIKLSMLTKKACLHLNPGG
TCVSIGYGYADRASESIIGAIARQFKFSRVCKPKSSHEETEVLFVFIGYD
RKARTHNPYKLSSTLTNIYTGSRLHEAGCAPSYHVVRGDIATATEGVIIN
AANSKGQPGGGVCGALYKKFPESFDLQPIEVGKARLVKGAAKHIIHAVGP
NFNKVSEVEGDKQLAEAYESIAKIVNDNNYKSVAIPLLSTGIFSGNKDRL
TQSLNHLLTALDTTDADVAIYCRDKKWEMTLKEAVARREAVEEICISDDS
SVTEPDAELVRVHPKSSLAGRKGYSTSDGKTFSYLEGTKFHQAAKDIAEI
NAMWPVATEANEQVCMYILGESMSSIRSKCPVEESEASTPPSTLPCLCIH
AMTPERVQRLKASRPEQITVCSSFPLPKYRITGVQKIQCSQPILFSPKVP
AYIHPRKYLVETPPVEETPESPAENQSTEGTPEQPALVNVDATRTRMPEP
IIIEEEEEDSISLLSDGPTHQVLQVEADIHGSPSVSSSSWSIPHASDFDV
DSLSILDTLDGASVTSGAVSAETNSYFARSMEFRARPVPAPRTVFRNPPH
PAPRTRTPPLAHSRASSRTSLVSTPPGVNRVITREELEALTPSRAPSRSA
SRTSLVSNPPGVNRVITREEFEAFVAQQQXRFDAGAYIFSSDTGQGHLQQ
KSVRQTVLSEVVLERTELEISYAPRLDQEKEELLRKKLQLNPTPANRSRY
QSRRVENMKAITARRILQGLGHYLKAEGKVECYRTLHPVPLYSSSVNRAF
SSPKVAVEACNAMLKENFPTVASYCIIPEYDAYLDMVDGASCCLDTASFC
PAKLRSFPKKHSYLEPTIRSAVPSAIQNTLQNVLAAATKRNCNVTQMREL
PVLDSAAFNVECFKKYACNNEYWETFKENPIRLTEENVVNYITKLKGPKA
AALFAKTHNLNMLQDIPMDRFVMDLKRDVKVTPGTKHTEERPKVQVIQAA
DPLATADLCGIHRELVRRLNAVLLPNIHTLFDMSAEDFDAIIAEHFQPGD
CVLETDIASFDKSEDDAMALTALMILEDLGVDAELLTLIEAAFGEISSIH
LPTKTKFKFGAMMKSGMFLTLFVNTVINIVIASRVLRERLTGSPCAAFIG
DDNIVKGVKSDKLMADRCATWLNMEVKIIDAVVGEKAPYFCGGFILCDSV
TGTACRVADPLKRLFKLGKPLAVDDEHDDDRRRALHEESTRWNRVGILPE
LCKAVESRYETVGTSIIVMAMTTLASSVKSFSYLRGAPITLYG Amino
acid sequence corresponding to nsp3 is underlined.

In this example, amino acid sequence of nsp3 which is corresponding from 1330-1886 in SEQ ID NO: 28 was replaced with the sequence shown below. The underlined sequence was different from SEQ ID NO: 28.

(SEQ ID NO: 29)
APSYHVVRGDIATATEGVIINAANSKGQPGGGVCGALYKKFPESFDLQPI
EVGKARLVKGAAKHIIHAVGPNFNKVSEVEGDKQLAEAYESIAKIVNDNN
YKSVAIPLLSTGIFSGNKDRLTQSLNHLLTALDTTDADVAIYCRDKKWEM
TLKEAVARREAVEEICISDDSSVTEPDAELVRVHPKSSLAGRKGYSTSDG
KTFSYLEGTKFHQAAKDIAEINAMWPVATEANEQVCMYILGKSMSSIRSK
CPVEESEASTPPSTLPCLCIHAMTPERVQRLKASRPEQITVCSSFPLPKY
RITGVQKIQCSQPILFSPKVPAYIHPRKYLVETPPVDETPEPSAENQSTE
GTPEQPPLITEDETRTRTPEPIIIEEEEEDSISLLSDGPTHQVLQVEADI
HGPPSVSSSSWSIPHASDFDVDSLSILDTLEGASVTSGATSAETNSYFAK
SMEFLARPVPAPRTVFRNPPHPAPRTRTPSLAPSRACSRTSLVSTPPGVN
RVITREELEALTPSRTPSRSVSRTSLVSNPPGVNRVITREEFEAFVAQQQ
XRFDAGA

### EXAMPLE 3

### Preparation of Self-amplifying RNA (saRNA)

Plasmids with influenza HA variant sequences placed downstream from a T7 promoter were linearized by digestion with the Nrul or BspQ1 restriction enzymes at 37° C. or 50° C. for 3 hours. The linearized plasmid was then purified using the Wizard Plus SV Miniprep DNA Purification System (Promega), and saRNA was transcribed in vitro using the T7 RiboMAX Express Large-Scale RNA Production System (Promega). After DNase treatment, the saRNA was purified with RNeasy midi kit (Qiagen), and subsequently modified by the addition of a 7-methylguanosine cap with the Vaccinia Capping System (New England Biolabs [NEB]) using the NEB Capping protocol (NEB, M20280). After purification of the capped saRNA using the Monarch kit (NEB).

### Western Blotting

HEK293T cells were transfected with 0.5 of saRNA using lipofectamine (Promega). Cells were collected 18 hours post transfection, lysed in cell lysis buffer (Cell Signaling Technology) and fractionated by SDS-PAGE (Any kD acrylamide gel, Bio-Rad). Proteins were detected by western blotting using anti-IAV H1N1 (A/California/07/2009) hemagglutinin, rabbit polyclonal antibody (1:5000 dilution; Sino Biological) and horseradish peroxidase-conjugated mouse anti-rabbit IgG (1:2000; Santa Cruz Biotechnology). The protein bands were visualized by enhanced chemiluminescence using ChemiDoc™ XRS+(Bio-Rad) and an Image Lab™ Software (Bio-Rad).

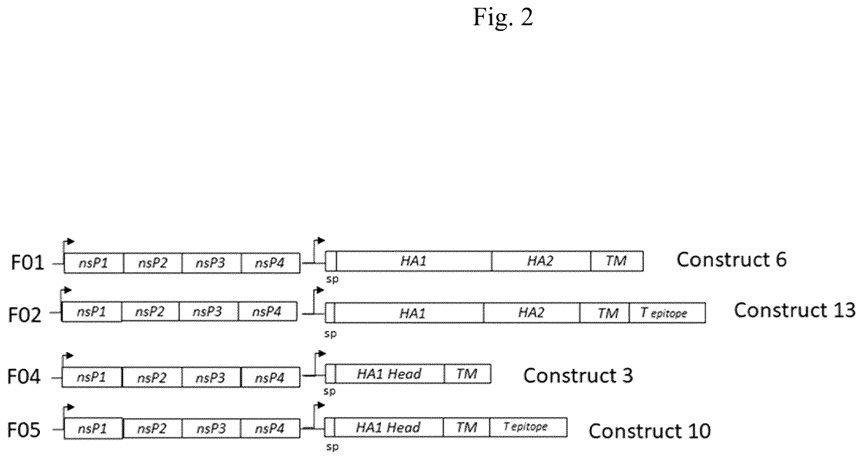

Results for plasmid vectors having constructs 6, 13, 3 and 10 as the gene of interest are shown in FIG. 3. As shown in FIG. 2, those constructs are corresponding to F01, F02, F04 and F05 respectively. The result of the Western Blot assay indicates that the cells transfected with saRNA-Flu variants express influenza antigen with the expected molecular weight.

### Flow Cytometric Analysis

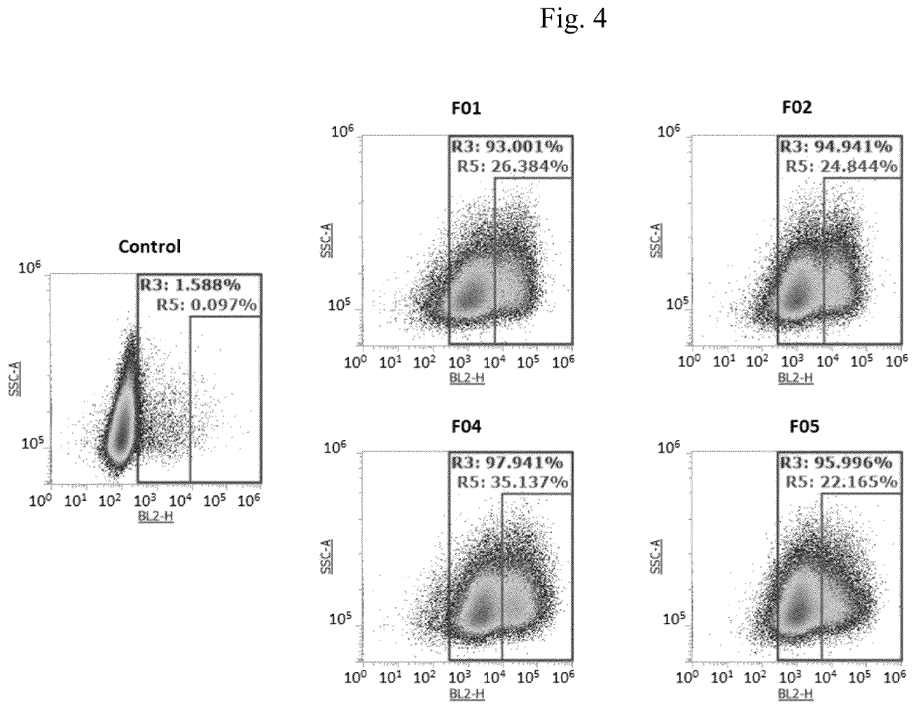

For the analysis of cell surface proteins, the transfected HEK293T cells were collected and washed with PBS, and stained with an anti-IAV H1N1 (A/California/07/2009) hemagglutinin, rabbit polyclonal antibody and donkey anti-rabbit IgG secondary PE (1:200 dilution; BioLegend). The levels of surface proteins were evaluated using an Attune acoustic focusing cytometer (applied biosystems). As a control, HEK293T cells without transfection were used. Results are shown in FIG. 4. In FIG. 4, R3 fractions contain cells positive for the antigen and R5 fractions contain cells highly positive for the antigen among the R3 fraction.

The flow cytometric analysis indicates that influenza antigens were expressed on the surface of the cells transfected with saRNA-Flu variants. In a previous study, the proportion of cells in the R5 fraction was correlated with immunogenicity against the antigen in an animal model test.

### EXAMPLE 4

### Preparation of Alphavirus Replicon Particles

10 μg of the full-length replicon plasmid prepared in Example 2, 1 μg of VEEV Env expression plasmid and 1 μg of VEEV Capsid NLS mutant (or 1 μg VEEV Capsid expression plasmid) was transfected into HEK293T cells. The supematant was harvested 48-96 hours after transfection. The replicon particle was purified by using an ion exchange column. HEK293T or Vero cells were infected with dilutions of the purified particle preparation to determine the infectious titer. The purified replicon particles are used for producing antigens for diagnosis and vaccination.

### EXAMPLE 5

### Preparation of mRNA or Self-amplifying RNA (saRNA) Encapsulated in Lipid Nanoparticles (LNP)

The plasmid vectors comprising the DNA sequence encoding construct 1-14 as the gene of interest prepared in Example 2was used. The plasmid vector was linearized and used as the template. T7 in vitro transcription was conducted based on protocols provided by the T7 transcription kit (RiboMax™ Express Large Scale RNA production System, Promega, (WI USA)). The linear DNA template was mixed with T7 enzyme and rNTPs to synthesize RNA. For the synthesis of RNA containing a modified nucleotide, a modified NTP such as 5-methyl-cytidine and N1-methyl-psudouridine triphosphate was added to the in vitro transcription reaction mixture. The purified RNA product was capped using vaccinia capping enzyme to give self-amplifying RNA.

The obtained mRNA or saRNA was encapsulated in lipid nanoparticles to give mRNA or saRNA particles.

What is claimed is:

1. An isolated polynucleotide, which encodes a polypeptide comprising an antigen protein fused to a signal sequence and a transmembrane domain, and optionally to a ferritin.

2. The polynucleotide of claim 1, the antigen protein is an influenza protein.

3. The polynucleotide of claim 1, wherein the influenza protein is an influenza-virus hemagglutinin 1(HA1) and/or an influenza-virus hemagglutinin 2(HA2).

4. The polynucleotide of claim 1, wherein the influenza protein is a head region of HA1.

5. The polynucleotide of claim 1, wherein the ferritin is derived from Helicobacter pylori or a Helicobacter pylon-bullfrog hybrid ferritin.

6. The polynucleotide of claim 1, which is further encodes an alphavirus non-structural proteins nsp1, nsp2, nsp3 and nsp4.

7. The polynucleotide of claim 1, wherein the transmembrane domain and/or signal sequence is fused to the antigen protein by a linker.

8. The polynucleotide of claim 1, wherein the polynucleotide is RNA.

9. The polynucleotide of claim 1, wherein the polynucleotide is DNA.

10. A vector comprising the polynucleotide of claim 1.

11. The vector of claim 10, which comprises a promoter, 5′ UTR, polynucleotide encoding alphavirus non-structural proteins nsp1, nsp2, nsp3 and nsp4, SG promoter, a gene of interest encoding the polypeptide comprising an antigen protein fused to a signal sequence and a transmembrane domain, 3′UTR and poly A tail.

12. A vaccine composition comprising the polynucleotide of claim 1 and a pharmaceutically acceptable carrier.

13. The vaccine composition of claim 12, wherein the pharmaceutically acceptable carrier is a delivery vehicle.

14. The vaccine composition of claim 13, wherein the delivery vehicle is a particle consisting of one or more alphavirus structural proteins or a lipid delivery system.

15. A method of inducing immunomodulation in a subject, comprising administering an immunologically effective amount of the vaccine composition of claim 12 to the subject in need thereof.

16. A method of treating, preventing and/or immunizing against an antigen in a subject, comprising administering an effective amount of the vaccine composition of claim 12 to the subject in need thereof.

17. A polypeptide comprising an antigen protein fused to a signal sequence and a transmembrane domain, and optionally to ferritin.


SEQUENCE LISTING (29 sequences)

SEQ ID NO: 1 - protein, 405 aa, Synthetic construct
MKAILVVLLYTFATANALHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 2 - protein, 278 aa, Synthetic construct
MKAILVVLLYTFATANALHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGGVKLESMGIYQILAIYSTVASSLVLLVSLGAISFWMCSNGSLQCRICI

SEQ ID NO: 3 - protein, 278 aa, Synthetic construct
MKAILVVLLYTFATANALHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGGVKLESTRIYQILAIYSTVASSLVLVVSLGAISFWMCSNGSLQCRICI

SEQ ID NO: 4 - protein, 401 aa, Synthetic construct
MFVFLVLLPLVSSLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 5 - protein, 274 aa, Synthetic construct
MFVFLVLLPLVSSLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGGVKLESMGIYQILAIYSTVASSLVLLVSLGAISFWMCSNGSLQCRICI

SEQ ID NO: 6 - protein, 566 aa, Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESMGIYQILAIYSTVASSLVLLVSLGAISFWMCSNGSLQCRICI

SEQ ID NO: 7 - protein, 692 aa, Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 8 - protein, 418 aa, Synthetic construct
MKAILVVLLYTFATANALHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSAKFVAAWTLKAAA

SEQ ID NO: 9 - protein, 291 aa, Synthetic construct
MKAILVVLLYTFATANALHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGGVKLESMGIYQILAIYSTVASSLVLLVSLGAISFWMCSNGSLQCRICIAKFVAAWTLKAAA

SEQ ID NO: 10 - protein, 291 aa, Synthetic construct
MKAILVVLLYTFATANALHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGGVKLESTRIYQILAIYSTVASSLVLVVSLGAISFWMCSNGSLQCRICIAKFVAAWTLKAAA

SEQ ID NO: 11 - protein, 414 aa, Synthetic construct
MFVFLVLLPLVSSLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSAKFVAAWTLKAAA

SEQ ID NO: 12 - protein, 287 aa, Synthetic construct
MFVFLVLLPLVSSLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGGVKLESMGIYQILAIYSTVASSLVLLVSLGAISFWMCSNGSLQCRICIAKFVAAWTLKAAA

SEQ ID NO: 13 - protein, 579 aa, Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESMGIYQILAIYSTVASSLVLLVSLGAISFWMCSNGSLQCRICIAKFVAAWTLKAAA

SEQ ID NO: 14 - protein, 705 aa, Synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKAKFVAAWTLKAAA

SEQ ID NO: 15 - protein, 326 aa, Alphainfluenzavirus influenzae
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQS

SEQ ID NO: 16 - protein, 174 aa, Alphainfluenzavirus influenzae
GLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEID

SEQ ID NO: 17 - protein, 13 aa, Severe acute respiratory syndrome-related coronavirus
MFVFLVLLPLVSS

SEQ ID NO: 18 - protein, 17 aa, Alphainfluenzavirus influenzae
MKAILVVLLYTFATANA

SEQ ID NO: 19 - protein, 213 aa, Alphainfluenzavirus influenzae
LHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAG

SEQ ID NO: 20 - protein, 48 aa, Alphainfluenzavirus influenzae
GVKLESMGIYQILAIYSTVASSLVLLVSLGAISFWMCSNGSLQCRICI

SEQ ID NO: 21 - protein, 48 aa, Alphainfluenzavirus influenzae
GVKLESTRIYQILAIYSTVASSLVLVVSLGAISFWMCSNGSLQCRICI

SEQ ID NO: 22 - protein, 173 aa, Synthetic construct
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 23 - protein, 13 aa, Synthetic construct
AKFVAAWTLKAAA

SEQ ID NO: 24 - DNA, 35 bp, Synthetic construct
  source 1..35: mol_type = other DNA
cctgaatggactacgacatagtctagtccgccaag

SEQ ID NO: 25 - DNA, 44 bp, Synthetic construct
  source 1..44: mol_type = other DNA
ataggcggcgcatgagagaagcccagaccaattacctacccaaa

SEQ ID NO: 26 - DNA, 125 bp, Synthetic construct
  source 1..125: mol_type = other DNA
gcgatcgcatacagcagcaattggcaagctgcttacatagaactcgcggcgattggcatgccgccttaaaatttttattttatttttcttttcttttccgaatcggattttgtttttaatatttc

SEQ ID NO: 27 - DNA, 55 bp, Synthetic construct
  source 1..55: mol_type = other DNA
aaaaaaaaaaaaaaaaaaaaaaaaaaaaaaaaaaaaaaaaaaaaaaaaaaaaaaa

SEQ ID NO: 28 - protein, 2493 aa, Venezuelan equine encephalitis virus
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDIQEAKCAADEAKEVREAEELRAALPPLAADFEEPTLEADVDLMLQEAGAGSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKRMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKILTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKKVDWLSDQPEATFRARLDLGIPGDVPKYDIVFINVRTPYKYHHYQQCEDHAIKLSMLTKKACLHLNPGGTCVSIGYGYADRASESIIGAIARQFKFSRVCKPKSSHEETEVLFVFIGYDRKARTHNPYKLSSTLTNIYTGSRLHEAGCAPSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVEETPESPAENQSTEGTPEQPALVNVDATRTRMPEPIIIEEEEEDSISLLSDGPTHQVLQVEADIHGSPSVSSSSWSIPHASDFDVDSLSILDTLDGASVTSGAVSAETNSYFARSMEFRARPVPAPRTVFRNPPHPAPRTRTPPLAHSRASSRTSLVSTPPGVNRVITREELEALTPSRAPSRSASRTSLVSNPPGVNRVITREEFEAFVAQQQXRFDAGAYIFSSDTGQGHLQQKSVRQTVLSEVVLERTELEISYAPRLDQEKEELLRKKLQLNPTPANRSRYQSRRVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATADLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAVDDEHDDDRRRALHEESTRWNRVGILPELCKAVESRYETVGTSIIVMAMTTLASSVKSFSYLRGAPITLYG

SEQ ID NO: 29 - protein, 557 aa, Venezuelan equine encephalitis virus
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGKSMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQXRFDAGA